(19) United States
(12) Patent Application Publication
(10) Pub. No.: US 2015/0327861 A1
(43) Pub. Date: Nov. 19, 2015

(54) RADIOPAQUE SUTURE

(71) Applicant: S. Jackson, Inc., Alexandria, VA (US)

(72) Inventors: Janko JACKSON, Alexandria, VA (US); Stephen L. JACKSON, Alexandria, VA (US); Crayton G. TONEY, Arlington, VA (US)

(73) Assignee: S. Jackson, Inc., Alexandria, VA (US)

(21) Appl. No.: 14/281,274

(22) Filed: May 19, 2014

(51) Int. Cl. A61B 17/06; A61L 17/00

(52) U.S. Cl. CPC A61B 17/06166; A61B 17/06004; A61L 17/00; A61B 2017/06019; A61B 2017/00526

(57) ABSTRACT

A polyamide suture includes an elongate core formed of multiple twisted and heat set filaments formed of a first polyamide material and a sheath formed of a second polyamide material surrounding the core along its length, the second polyamide material having dispersed therein non-absorbable radiopaque nanoparticles comprising 15-25% by weight of the second polyamide material. The melting point of the first polyamide material is at least 30° C. greater than the melting point of the second polyamide material. The core may also include or be formed of previously extruded bundles of first polyamide filaments overcoated with a second polyamide sheath. The suture is made by coextruding the core and a molten organic material formed of the second polyamide material and dispersed radiopaque nanoparticles. Desirably, the first polyamide material is Polyamide 66 and the second polyamide material is Polyamide 6.

### FIELD OF THE INVENTION

The present invention relates to polyamide radiopaque sutures and a method for making such sutures and, more particularly, to non-absorbable polyamide sutures comprising a multitude of polyamide filaments encased within a polyamide sheath which includes radiopaque material distributed throughout the sheath, and a method of making such sutures.

### BACKGROUND OF THE INVENTION

According to the U.S. Pharmacopeia (USP), a nonabsorbable surgical suture is a flexible strand of material that is suitably resistant to the action of living mammalian tissue. It may be in either monofilament or multifilament form. If the latter, the individual filaments may be combined by spinning, twisting, braiding or any combination thereof. It may be either sterile or nonsterile and its diameter and knot pull tensile strength must be within the limits prescribed by the USP.

In many surgical procedures it is important or advantageous to be able to monitor the condition of sutures installed during surgery or to monitor the condition of an internal scar previously sutured. Inasmuch as scars and all but metal sutures are transparent to X-ray photography, and the use of metal sutures is frequently undesirable, only the use of sutures having radiopaque properties provides the needed monitoring capability. However, radiopaque non-metallic sutures are currently commercially unavailable.

Historically, efforts at making a non-metallic radiopaque suture focused on absorbable monofilament sutures and sought to make them radiopaque by coating them over their outer surface with a radiopaque free metal or coating or impregnating the suture with radiopaque beads or mounting radiopaque clip members at spaced apart intervals along the suture surface. See, for example, U.S. Pat. No. 3,194,239—Sullivan. However, attempts at making a non-metallic suture radiopaque along its entire length, such as by coating, were found to weaken the resultant suture such that it no longer could meet USP standards for suture tensile or knot strength. Moreover, the coating process produced inconsistent suture diameters such that the suture average diameter could not satisfy USP limits for its intended size. In addition, it was found that applying a coating to its outer surface did not permit the suture to exhibit suitable abrasion resistance, making it difficult or impossible to swage onto a needle. Radiopaque sutures utilizing beads, clips or other markers spaced at discrete points along the suture presented problems of needle attachment and invited an increased likelihood of tissue inflammation. For many of these same reasons, it has proven to be difficult to make multifilament non-metallic radiopaque sutures. In addition multifilament sutures present difficulties in obtaining long term sheath to multifilament adherence, particularly when the multifilament polymer component is markedly chemically different from the shell polymer component.

Accordingly, there exists a need for non-metallic radiopaque sutures and a method for making such sutures, particularly for non-absorbable surgical sutures comprising a core bundle of twisted or braided filaments having an outer sheath which includes radiopaque material distributed throughout the sheath and a method of making such sutures.

### SUMMARY OF THE INVENTION

In accordance with one aspect, the present invention provides a polyamide suture comprising an elongate core formed of multiple twisted and heat set filaments formed of a first polyamide material and a sheath surrounding the core along its length, the sheath comprises a second polyamide material, said second polyamide material having dispersed therein non-absorbable radiopaque nanoparticles, the radiopaque nanoparticles comprising 15-25% by weight of said second polyamide material. The melting point of the first polyamide material is at least 30° C. greater than the melting point of the second polyamide material.

In another aspect of the invention, there is provided a polyamide suture wherein the melting point of the first polyamide material is 30° -50° C. greater than the melting point of the second polyamide material.

In still another aspect of the invention, there is provided a polyamide suture wherein the first polyamide material is Polyamide 66 and the second polyamide material is Polyamide 6.

In yet another aspect of the invention, there is provided a polyamide suture wherein the radiopaque nanoparticles are smaller than 1000 nm, desirably in the range from 50 to 100 nm, and comprise about 20% by weight of the second polyamide material.

In still another aspect of the invention, there is provided a polyamide suture having an elongate core comprising twisted or braided and heat set individual filaments of a first polyamide material, or previously extruded bundles of filaments of a first polyamide material, each said bundle comprising multiple twisted or braided first polyamide filaments, heat set and overcoated with a second polyamide sheath, or a mixture of said filaments and said previously extruded bundles.

In another aspect of the invention, there is provided a method of making a polyamide suture comprising forming an elongate core comprising: filaments of a first polyamide material, and twisting and heat setting said filaments, or previously extruded bundles of filaments of a first polyamide material, each bundle comprising multiple twisted or braided first polyamide filaments, heat set and overcoated with a second polyamide sheath, or a mixture of said filaments and said previously extruded bundles, said core having a generally round cross-section suitable for coextrusion; coextruding said core and a molten organic material comprising said second polyamide material having dispersed therein non-absorbable radiopaque nanoparticles to form a sheath surrounding said core along its length, said radiopaque particles comprising 15-25% by weight of the second polyamide material; and selecting the first polyamide material to have a melting point at least 30° C. greater than the melting point of the second polyamide material.

In still another aspect of the invention, there is provided a method of making a polyamide suture including the step of selecting said first polyamide material to have a melting point 30°-50° C. greater than the melting point of said second polyamide material.

In yet another aspect of the invention, there is provided a method of making a polyamide suture including the step of selecting said first polyamide material as Polyamide 66 and said second polyamide material as Polyamide 6.

In another aspect of the invention, there is provided a method of making a polyamide suture including the step of dispersing radiopaque nanoparticles smaller than 1000 nm, desirably in the range from 50 to 100 nm, in an organic material comprising the second polyamide material prior to coextruding said core with said organic material, wherein said radiopaque nanoparticles comprise about 20% by weight of said second polyamide material.

### BRIEF DESCRIPTION OF THE DRAWINGS

FIG. 1 is a cross-sectional view of an extrusion coated multifilament radiopaque suture in accordance with the present invention wherein the suture core comprises a multitude of polyamide filaments twisted or braided together and heat set.

FIG. 2 is a cross-sectional view of an extrusion coated multifilament radiopaque suture in accordance with the present invention wherein the suture core comprises a mixture of polyamide filaments and previously extruded bundles comprising a multitude of individual polyamide filaments twisted or braided together, heat set and overcoated with a polyamide sheath.

FIG. 3 is a schematic view of apparatus for manufacture of the radiopaque sutures of the present invention.

### DETAILED DESCRIPTION OF THE INVENTION

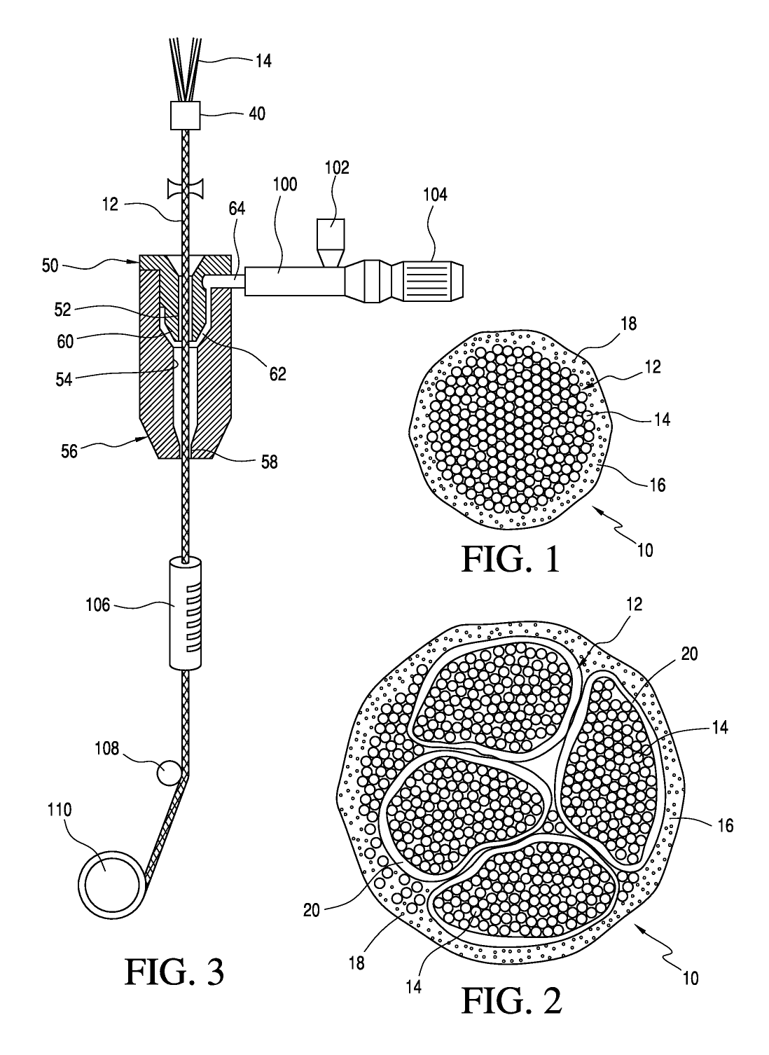

The present invention provides a unique polyamide radiopaque suture and a method for manufacturing this suture. The suture provided by the present invention is a significant improvement over previously available sutures in that it is radiopaque along its entire length, exhibits superior abrasion resistance and conforms to all of the USP strength requirements for its size. The suture comprises a core of a plurality of twisted polyamide filaments which are heat set to provide a stable and approximately round cross section suitable to be overcoated by a sheath of polyamide applied by polymer coextrusion techniques, such as cross-head extrusion. According to the invention, prior to extrusion of the sheath, nanoparticle radiopaque materials, such as tantalum particles less than 1000 nm, are blended into the sheath material to provide the desired radiopacity along the entire length of the suture. Referring to FIG. 1, there is shown a cross section of a suture 10 made according to the present invention after it has exited a cross-head die. Suture 10 comprises a core 12 formed of a multitude of polyamide fibers 14 and a polyamide sheath 16 having nanoparticle sized radiopaque material 18 dispersed throughout the thickness and length of sheath 16. Core 12 may be formed by (1) a multitude of individual polyamide filaments twisted or braided together and heat set or (2) previously extruded bundles of filaments, each bundle comprising a multitude of individual polyamide filaments twisted or braided together, heat set and overcoated with a polyamide sheath, the bundles being twisted together to form a core having the correct diameter for the suture being made or (3) a combination of individual filaments as in (1) and previously extruded bundles of filaments as in (2). FIG. 1 illustrates a core 12 formed of individual filaments 14 encased within sheath 16 containing dispersed radiopaque material 18 which has been extruded thereabout as will be discussed more fully hereinafter. FIG. 2 illustrates a core 12 formed of previously extruded bundles 20 of filaments 14 overcoated with a sheath 16, the core 12 being encased within sheath 16 which has been extruded thereabout. Optionally, core 12 may include, in addition to bundles 20, individual filaments 14 to act as a filler in some of the spaces formed between the bundles 20. The twisting and heat setting of filaments are important to provide a stable and approximately round core cross section for enabling overcoating via cross-head extrusion techniques. Optionally, adherence of the filaments may be improved, particularly where using incompatible materials in the filaments and the sheath, by coating the fibers in a well known manner in addition to twisting and heat setting.

Sutures require a unique combination of physical properties. They must be nonirritating, flexible and exhibit high tensile strength and knot strength. Additionally, sutures must retain their physical properties after conventional processing such as dyeing, sterilization and resterilization. Some elasticity is required in the final suture structure to obtain the required knot strength and other properties to allow the suture to meet USP specifications. The twist level and number of filaments helps determine the final suture properties and size. Thus, the larger the filament size and the greater the number of filaments the larger the final suture diameter and total strength.

The sheath-core configuration of the multifilament sutures of the present invention is only possible if there is a difference in melting points (m.p.) between the polyamide material of the filaments in the core and the polyamide material of the sheath material, with the polyamide core filaments having a higher melting temperature than the polyamide sheath material. The temperature differential must be enough (i.e., at least 30° C., preferably 30° C. to 50° C.) to assure that the filaments in the core do not melt during over coating of the sheath. In this regard, large temperature differentials would seem to allow easier processing. However, there must be good adhesion between the core and the sheath and, often, polymers having large melting point differences are not sufficiently compatible to avoid problems such as sheath splitting or core-sheath separation.

Preferably, the suture of the present invention is made with Polyamide 66 (polyhexamethylene adipamide) core filaments (m.p. 255° C.) and Polyamide 6 (polycaprolactam) sheath (mp 220° C.). It has been found that good compatibility is obtained between these polyamides to obtain the desired adhesion to keep the sheath and filaments adhered as a single structure. Moreover, Polyamide 66 and Polyamide 6 are currently in use in non-absorbable sutures marketed under the trademark SUPRAMID® by S. Jackson, Inc. of Alexandria, VA. and the trademarks SUPRAMID® and BRAUNAMID by B. Braun Melsungen AG of Tuttlingen, Germany and are known to be compatible as suture components. Subject to the requirements of melting point differential and other suture-desirable properties, such as flexibility, any nontoxic, substantially nonirritating polyamide may be used in the practice of the invention. These include, but are not limited to, homopolymers such as Polyamide 69, Polyamide 610, Polyamide 612, Polyamide 11, Polyamide 12, Polyamide 61, and copolymers of the foregoing.

Sutures made in accordance with the present invention have a generally round cross section. However, as will be appreciated from the manner of their manufacture, due to gravity and the necessity to pass the filaments around rolls, there is some deformation. When the sutures are made using core filaments of Polyamide 66 and a sheath of Polyamide 6, these sutures meet all USP specifications of average diameter and knot pull tensile strength for sutures of their size. and class. The number and size of Polyamide 66 filaments is selected according to the suture size desired.

The selected radiopaque material is in the nanoparticle size range and is desirably spherical in shape but can have irregular surfaces due to its production process. Desirably, it is less than 1000 nm. The size is, in part, dependent upon the size of the suture and the thickness of the sheath. The particles must be small enough that they don't interfere with the overcoating process or cause splits or other failure in the sheath. Preferably, the particles are in the range 50 to 100 nm. The preferred radiopaque material is nanoparticulate tantalum or tantalum oxide, which are known to be highly bioinert and to possess high radiographic density, allowing them to be used at relatively lower concentrations. Other highly desirable radiopaque materials include titanium, zirconium, silver, bismuth and platinum in elemental, salt or oxide form. Consistent with the foregoing criteria, other radiopaque materials may be used as well. However, inasmuch as the concentration of radiopaque particles in the sheath is a function of the radiopacity of the particle selected, some radiopaque materials are unsuitable due to the high concentrations which would be required to achieve the desired radiopacity. Thus, for example, barium sulfate or oxide would be unsuitable for use in sutures for this reason. Also, some additives may not meet FDA regulations for medical devices. The radiopaque particles are, typically, dispersed within the sheath polymer, for example, by blending the particles with powder or pellets of the sheath polymer prior to or during extrusion. For tantalum nanoparticles, a concentration of about 15% to 25% of the sheath polymer by weight appears to be satisfactory, desirably about 20% by weight of the sheath polymer.

The method of making the suture 10 of the present invention will be described hereinafter in connection with a USP 3-0 suture which has a core formed of twisted and heat set Polyamide 66 filaments and a sheath of Polyamide 6 having radiopaque tantalum nanoparticles uniformly dispersed within the Polyamide 6 extruded over the core to a desired thickness. It will be appreciated that substantially the same method can be practiced on other polyamides, as hereinbefore discussed, and using other radiopaque nanoparticles with only minor changes as are well known to those skilled in the art.

Initially, a core of Polyamide 66 filaments is formed by introducing multiple individual Polyamide 66 filaments into a conventional braider at a setting determined to provide a moderately tight twisted core having a desired diameter to produce, after overcoating a Polyamide 6 sheath, a final suture meeting USP specifications for USP Size 3-0 sutures The braider is operated at a temperature in the range of 140-200° C. in order to heat set the twisted core as it is formed. Alternatively, several bundles of previously extruded filaments twisted or braided together, heat set and overcoated with Polyamide 6 may be twisted together to form a core having the desired diameter. In the latter alternative, individual filaments may be used to fill the spaces between the bundles and to facilitate obtaining a round cross section. The resulting twisted, heat set core can be wound upon a roll and stored prior to the step of overcoating or can be immediately directed to a co-extrusion apparatus having a cross-head extruder tip and die assembly for impregnation and continuous coating of the core with a molten Polyamide 6/tantalum particle-containing sheath as hereinafter described.

In the overcoating process a twisted, heat set core of Polyamide 66 filaments having a generally circular cross section is guided by a member to bring the core into a cross-head tip and extrusion die assembly for co-extruding the core with a molten thermoplastic Polyamide 6 in which is uniformly distributed nanoparticles of radiopaque tantalum. The core is continuously fed to a cross-head tip of the assembly. The molten Polyamide 6/tantalum material is continuously supplied by an extrusion apparatus to a chamber in the cross-head tip, wherein the Polyamide 6/tantalum material uniformly distributes about the core prior to co-extrusion by passage of the core and the molten resin material through a round co-extrusion die of the assembly. Following extrusion, the Polyamide 6/tantalum extrusion coated core is transported through a cooling device downstream from the die assembly in which cooling water is sprayed into the interior of the cooling device for cooling the coated suture material prior to being wound onto a spindle rotated by a motor.

Referring to FIG. 3, multiple Polyamide 66 filaments 14 are fed to a braider 40 operating at a temperature of about 160° C. to form a twisted, heat set core 12 of Polyamide 66 filaments having a generally circular cross section. The twisted core 12 is continuously fed and guided to the cross-head extruder tip 50, transported through a cross head passage 52, then concentrically through a die head cavity 54 in the die assembly 56, and extruded through an outlet orifice 58 of the die assembly 56. The cross head passage 52 extends through a frusto-conical end 60 of the cross-head extruder tip 50, where the passage 52 is surrounded by a concentric chamber 62 filled with the molten thermoplastic Polyamide 6/tantalum resin mix under pressure. The resin mix under pressure fills the chamber 62 and surrounds the concentric cross-head end 60. The chamber 62 communicates with a feed duct 64 into which is continuously fed the molten Polyamide 6 resin/tantalum resin mix from an extruder 100.

Extruder 100 has an input hopper 102 into which is continuously supplied meltable pellets or powder of thermoplastic Polyamide 6 and nanoparticles of tantalum. The Polyamide 6 and tantalum particles are admixed, heated and driven under pressure of a drive screw in extruder 100 to the feed duct 64. Alternatively, if available, pellets of a previously extruded Polyamide 6/tantalum nanoparticle mix may be supplied to hopper 102. A motor 104 is provided to turn the screw drive. The cross-head end 60 and the metal material surrounding chamber 62 are at an elevated melting temperature of the molten thermoplastic Polyamide 6 resin to maintain continuous melt flow. When the core in the die head cavity 54 comes into contact with the molten Polyamide 6 resin/tantalum mix under pressure, the mix distributes uniform radial pressure on the entire periphery of the core as soon as contact is established. As a result the core is impregnated and continuously coated by the Polyamide 6 resin/tantalum mix. Due to the pressure and/or viscosity of the resin material and the velocity at which the core passes through the device, the thickness of the area within the core penetrated by the Polyamide 6 resin/tantalum mix can be controlled for a given sheath polymer. The thickness of the impregnated area of the core also depends on the degree of chemical compatibility which is likely to exist between the molten coating material and the filaments constituting the core. If the level of compatibility is high, as it is with Polyamide 66 filaments and Polyamide 6 sheathing, the filaments are easily wetted by the organic material.

As the cavity 54 progressively narrows in a direction toward the round outlet orifice 58, the core and Polyamide 6 resin/tantalum mix are co-extruded by transport through the round outlet orifice 58. Outlet orifice 58 is machined with a round orifice to distribute uniform pressure of the molten Polyamide 6 resin/tantalum mix over the surface of the core and is dimensioned to apply a uniform coating of Polyamide 6 resin/tantalum mix to form a round cross-section suture having the desired diameter. Following coextrusion, the extrusion coated core is cooled in cooling device 106 downstream from the die assembly 56. The Polyamide 66 core 12 overcoated with the Polyamide 6/tantalum particle sheathing 16 which is substantially solidified, at least on the surface, passes over a guide member 108 before being wound onto a spindle 110 rotated by a motor (not shown).

While the present invention has been described in terms of specific embodiments thereof, it will be understood that no limitations are intended to the details of construction or design other than as defined in the appended claims.

1. A polyamide suture comprising:
an elongate core formed of multiple twisted and heat set filaments formed of a first polyamide material; and
a sheath surrounding said core along its length, said sheath comprising a second polyamide material, said second polyamide material having dispersed therein non-absorbable radiopaque nanoparticles, said radiopaque nanoparticles comprising 15-25% by weight of said second polyamide material,
the melting point of said first polyamide material being at least 30° C. greater than the melting point of said second polyamide material.

2. A polyamide suture, as claimed in claim 1, wherein said radiopaque nanoparticles are smaller than 1000 nm.

3. A polyamide suture, as claimed in claim 2, wherein said radiopaque nanoparticles are in the range from 50 to 100 nm.

4. A polyamide suture, as claimed in claim 1, wherein the melting point of said first polyamide material is 30°-50° C. greater than the melting point of said second polyamide material

5. A polyamide suture, as claimed in claim 1, wherein said radiopaque nanoparticles comprise about 20% by weight of said second polyamide material.

6. A polyamide suture, as claimed in claim 1, wherein said radiopaque nanoparticles are selected from the group consisting of tantalum, tantalum oxide, titanium, zirconium, silver, bismuth, platinum, and radiopaque oxides and salts thereof.

7. A polyamide suture, as claimed in claim 1, wherein said radiopaque nanoparticles are selected from tantalum and tantalum oxide.

8. A polyamide suture, as claimed in claim 1, wherein said first polyamide material is Polyamide 66 and said second polyamide material is Polyamide 6.

9. A polyamide suture, as claimed in claim 2, wherein said first polyamide material is Polyamide 66 and said second polyamide material is Polyamide 6.

10. A polyamide suture, as claimed in claim 9, wherein said radiopaque nanoparticles are selected from the group consisting of tantalum, tantalum oxide, titanium, zirconium, silver, bismuth, platinum, and radiopaque oxides and salts thereof.

11. A polyamide suture, as claimed in claim 10, wherein said radiopaque nanoparticles are selected from tantalum and tantalum oxide.

12. A polyamide suture, as claimed in claim 1, attached to a needle.

13. A polyamide suture, as claimed in claim 1, wherein said core comprises multiple individual polyamide filaments which are twisted or braided and heat set.

14. A polyamide suture, as claimed in claim 1, wherein said core comprises previously extruded bundles of filaments, each said bundle comprising multiple twisted or braided polyamide filaments, heat set and overcoated with a polyamide sheath.

15. A polyamide suture, as claimed in claim 14, wherein said core also comprises multiple individual polyamide filaments.

16. A method of making a polyamide suture, comprising:
forming an elongate core comprising
filaments of a first polyamide material, and twisting and heat setting said filaments, or
previously extruded bundles of filaments of a first polyamide material, each said bundle comprising multiple twisted or braided first polyamide filaments, heat set and overcoated with a second polyamide sheath, or
a mixture of said filaments and said previously extruded bundles,
said core having a generally round cross-section suitable for coextrusion;
coextruding said core and a molten organic material comprising said second polyamide material having dispersed therein non-absorbable radiopaque nanoparticles to form a sheath surrounding said core along its length, said radiopaque particles comprising 15-25% by weight of said second polyamide material; and
selecting said first polyamide material to have a melting point at least 30° C. greater than the melting point of said second polyamide material.

17. A method, as claimed in claim 16, including the step of attaching said suture to a needle.

18. A method, as claimed in claim 16, including the steps of:
injecting said molten organic material into a central duct of a device mounted in the manner of a cross-head at the end of an extruder;
moving said core along the axis of said central duct into contact with said organic material;
subjecting said core to a constant and uniform pressure; and
coextruding said core and said organic material through a circular outlet orifice of said duct for forming said sheath surrounding said core.

19. A method, as claimed in claim 16, including the step of dispersing radiopaque nanoparticles smaller than 1000 nm in said organic material prior to coextruding.

20. A method, as claimed in claim 16, including the step of dispersing radiopaque nanoparticles in the range 50 to 100 nm in said organic material prior to coextruding.

21. A method, as claimed in claim 16, including the step of selecting said first polyamide material to have a melting point 30°-50° C. greater than the melting point of said second polyamide material.

22. A method, as claimed in claim 16, including the step of coextruding said core with said organic material wherein said radiopaque nanoparticles comprise about 20% by weight of said second polyamide material.

23. A method, as claimed in claim 16, including the step of selecting said radiopaque nanoparticles in said organic material from the group consisting of tantalum, tantalum oxide, titanium, zirconium, silver, bismuth, platinum, and radiopaque oxides and salts thereof.

24. A method, as claimed in claim 16, including the step of selecting said radiopaque nanoparticles in said organic material from tantalum and tantalum oxide.

25. A method, as claimed in claim 16, including the step of selecting said first polyamide material as Polyamide 66 and said second polyamide material as Polyamide 6.
